SEQUENCE LISTING (4 sequences)

SEQ ID NO: 1 - protein, 103 aa, synthetic construct
TASLVEGVELTKYISDINNNTDGMYVVSSTGGVWRISRAKDYPDNVMTAEMRKIAMAAVLSGMRVNMCASPASSPNVIWAIELEAEGSGSGASQFFKDNCNRT

SEQ ID NO: 2 - protein, 103 aa, synthetic construct
NNTDGMYVVSSTGGVWRISRAKDYPDNVMTAEMRKIAMAAVLSGMRVNMCASPASSPNVIWAIELEAEGSGSGASQFFKDNCNRTTASLVEGVELTKYISDIN

SEQ ID NO: 3 - protein, 103 aa, synthetic construct
KDYPDNVMTAEMRKIAMAAVLSGMRVNMCASPASSPNVIWAIELEAEGSGSGASQFFKDNCNRTTASLVEGVELTKYISDINNNTDGMYVVSSTGGVWRISRA

SEQ ID NO: 4 - protein, 4 aa, synthetic construct
GSGS

(19) United States
(12) Patent Application Publication
(10) Pub. No.: US 2026/0055147 A1
(43) Pub. Date: Feb. 26, 2026

(54) RECOMBINANT PROTEIN AND PREPARATION PROCESS THEREOF, PROTEIN ADJUVANT FOR VACCINE COMPRISING RECOMBINANT PROTEIN, AND USE OF RECOMBINANT PROTEIN FOR PREVENTING RESPIRATORY DISEASE

(71) Applicant: National Tsing Hua University, Hsinchu (TW)

(72) Inventors: Shih-Che Sue, Hsinchu (TW); Suh-Chin Wu, Hsinchu (TW)

(21) Appl. No.: 18/953,141

(22) Filed: Nov. 20, 2024

(30) Foreign Application Priority Data
Aug. 22, 2024 (TW) 113131658

(51) Int. Cl. C07K 14/245; A61K 39/00; A61K 39/145

(52) U.S. Cl. CPC C07K 14/245; A61K 39/145; A61K 2039/543; A61K 2039/55516

(57) ABSTRACT

The present disclosure provides a recombinant protein and the preparation process thereof, a protein adjuvant for vaccines including the recombinant protein, and a use of the recombinant protein for preventing respiratory diseases. The recombinant protein of the present disclosure achieves the effect of preventing respiratory diseases through various efficacy experiments.

### CROSS-REFERENCE TO RELATED APPLICATION

This application claims priority of Taiwan patent application No. 113131658, filed on Aug. 22, 2024, the content of which is incorporated herein in its entirety by reference.

### STATEMENT REGARDING SEQUENCE LISTING

The sequence listing associated with this application is provided in text format in lieu of a paper copy and is hereby incorporated by reference into the specification. The name of the XML file containing the sequence listing is 113F0049-IE_Sequence_listing. The XML file is 5032 bytes; was created on Jan. 9, 2025.

### BACKGROUND OF THE INVENTION

### 1. Field of the Invention

The present invention relates to a recombinant protein and the preparation process thereof, a protein adjuvant for vaccine comprising the recombinant protein, and use of the recombinant protein for preventing respiratory disease.

### 2. The Prior Art

The respiratory mucosal immune system appears to be the major target of most human pathogens. Therefore, the infection might be prevented by a unique, potent pre-existing immune response in respiratory mucosal compartments. However, the major constraint in the development of mucosal vaccines is that antigens applied to mucosal membranes generally induce relatively weak immune responses, which is to avoid inducing severe responses to the numerous harmless antigens in the environment. To overcome the barrier, mucosal adjuvants must be integrated to into vaccine components to elevate the immune response. The incorporation of an appropriate adjuvant in vaccine formulations will assist in the stronger induction of protective immunity.

However, traditional vaccine adjuvants have the disadvantages of insufficient stability and cytotoxicity. In order to solve the above-mentioned problems, those skilled in the art urgently need to develop a novel, more effective and safe medicament for preventing respiratory immune disease for the benefit of a large group of people in need thereof.

### SUMMARY OF THE INVENTION

A primary objective of the present invention is to provide a recombinant protein, comprising a B₅ subunit of type II heat labile enterotoxin (hereinafter referred to as LTB) variant of Escherichia coli, wherein the LTB variant comprises at least one amino acid sequence which undergoes circular permutation (CP). The objective is to provide a method for modifying proteins using circular permutation and apply it to the development of vaccine adjuvants.

Another objective of the present invention is to provide a preparation method to produce the recombinant protein as described above, comprising performing circular permutation (CP) on a B₅ subunit of type II heat labile enterotoxin (LTB) of wild type Escherichia coli.

According to an embodiment of the present invention, the circular permutation is performed by disconnecting a corresponding loop of the B₅ subunit of type II heat labile enterotoxin (LTB) of wild type Escherichia coli and connecting original terminal ends by a GSGS linker.

According to an embodiment of the present invention, positions of residue 13-14, 31-32 or 52-53 of the LTB variant are disconnected to create new N- and C-termini.

According to an embodiment of the present invention, the residue 13-14 is Thr13-Thr14 (T13-T14, TT).

According to an embodiment of the present invention, the residue 31-32 is Asn31-Asn32 (N31-N32, NN).

According to an embodiment of the present invention, the residue 52-53 is Ala52-Lys53 (A52-K53, AK).

According to an embodiment of the present invention, the at least one amino acid sequence which undergoes circular permutation is selected from the group consisting of: SEQ ID NO:1, SEQ ID NO:2, SEQ ID NO:3, and a combination thereof.

According to an embodiment of the present invention, the recombinant protein may further comprise a 6×His-tag sequence, wherein the 6×His-tag sequence is conjugated in C-terminal of the LTB variants.

Another objective of the present invention is to provide a protein adjuvant for a vaccine, comprising the aforementioned recombinant protein.

Another objective of the present invention is to provide a method for preventing respiratory disease, comprising administering to a subject in need thereof a pharmaceutical composition comprising an effective amount of the aforementioned recombinant protein.

According to an embodiment of the present invention, the respiratory disease is selected from the group consisting of: influenza, severe acute respiratory syndrome (SARS), Coronavirus disease 2019 (COVID-19), and other disease transmitted through respiratory tract.

According to an embodiment of the present invention, the recombinant protein comprising the at least one amino acid sequence of a combination of SEQ ID NO: 1 and SEQ ID NO:2 does not have or reduces GD1a binding ability.

According to an embodiment of the present invention, the recombinant protein maintains ability to bind to TLR 2/1.

According to an embodiment of the present invention, a dosage form of the pharmaceutical composition is intramuscular injection, injection, nasal drops, spray, nasal spray or inhalation.

In summary, through the results illustrated in the following examples, the present invention uses the LTB protein scaffold to invent a new LTB circular recombinant proteins, and can use this circular recombination to develop new respiratory mucosal protein adjuvants, using circular permutation to design new LTB adjuvants with better structural stability, leveraging circular permutation to maintain the ability to activate TLR2/1 receptors to design novel LTB adjuvants to induce comparable or superior antibody activity. The use of circular permutation to design new LTB adjuvants can reduce the binding ability of GD1a gangliosides, thereby reducing cytotoxicity, designing new LTB adjuvants using circular permutation to induce better immune response stimulation.

The present invention utilizes the concept of circular permutation to design three LTB circular permutation (CP) variants and evaluate their GD1a binding ability and structural stability through nuclear magnetic resonance (NMR) technology. Among new LTB circular permutation variants, TT exhibits excellent structural stability and exhibits superior protein adjuvant activity. NN shows similar structural instability to WT and is easier to aggregate. TT and NN lose their binding ability to GD1a through circular permutation, but still retain their ability to activate TLR 2/1. AK has excellent structural stability, retains the binding ability to GD1a, and has low activation ability for TLR 2/1. The circular permutation variants of the present invention give LTB different protein adjuvant activities and can be used for different indications. It is expected to promote the future development of mucosal vaccines, especially for respiratory diseases such as influenza.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows GD1a binding sites of the B₅ subunit of type II heat labile enterotoxin (LTB) of Escherichia coli. There are two predicted GD1a binding packets on LTB surface (PDB code: 5G3L). The respective critical residues responsible for binding are depicted in FIG. 1.

FIG. 2 shows the strategy of using circular permutation (CP) to remove ligand binding. The present invention disconnects the protein peptide chain related to the ligand binding and connects the original terminal ends by a linker to newly design a GD1a-binding deficient LTB, while retaining the same structure as the original species and maintaining the ability to activate TLR 2/1.

FIG. 3 shows new design of LTB circular permutation constructs. We design three CPs, named TT, NN and AK by disconnecting the corresponding loop and connecting the original terminal ends by a GSGS linker.

FIGS. 4A and 4B show purification of LTB and the CP constructs. (4A) The FPLC gel filtration profiles of LTB and the CPs. The dotted lines indicate the presence of stable pentamer in LTB WT, TT, NN and AK. The misfolded LTB in WT and NN forms aggregates that elute before the pentamer peaks as indicated by the stars. (4B) SDS-PAGE analysis of purified LTB and the CPs pentamer in the presence and absence of β-mercaptoethanol, showing that excellent purity can be achieved after purification.

FIG. 5 is circular dichroism (CD) spectra at different temperatures demonstrating LTB CPs sharing the same secondary structural property as WT.

FIG. 6 is NMR HSQC Spectrums displaying the structural stability of LTB CPs. The LTB WT and NN show broadening resonances, indicating that the backbone is significantly dynamic and easily forms aggregates in aqueous solution. The LTB TT and AK show intense and dispersive resonances, indicating correct-folded structure and good structural stability.

FIG. 7 shows X-ray structures of LTB TT and AK and the comparison with the structure of LTB WT. (A) The alignment of the structure of LTB TT to the structure of WT shows great structural similarity. The CP sites of LTB TT are indicated by arrows where the representative GD1a main binding site is indicated by a circle. With the GD1a coordinate docking on the LTB TT structure, the GD1a binding packet is disrupted because of protein backbone disconnection. (B) The alignment of the structure of LTB AK to the structure of WT shows great structural similarity. The CP sites of LTB AK are indicated by arrows where the representative GD1a main binding site is indicated by a circle. With the GD1a coordinate docking on the LTB AK structure, the GD1a binding packet is intact that LTB AK remains the GD1a main binding site, but the secondary binding site is destroyed.

FIG. 8 shows NMR 1D ¹H titration experiment. Five NMR 1D ¹H spectra are compared, that are spectra for free LTB TT, free GD1a, TT to GD1a 1:1, TT to GD1a 1:2, TT to GD1a 1:3. The GD1a signals remain the same in the presence of LTB TT. In the subsequent titration, the signal intensity of GD1a is consistent with the dripping multiple, indicating the deficiency of LTB TT for binding GD1a.

FIG. 9 shows NMR 1D ¹H titration experiment. Five NMR 1D 1H spectra are compared, that are spectra for free LTB NN, free GD1a, NN to GD1a 1:1, NN to GD1a 1:2, NN to GD1a 1:3. The GD1a signals remain the same in the presence of LTB NN. In the subsequent titration, the signal intensity of GD1a is consistent with the dripping multiple, indicating the deficiency of LTB NN for binding GD1a.

FIG. 10A shows NMR HSQC spectra of LTB TT titrated with GD1a. The NMR sample containing 15N LTB TT was prepared in an NMR buffer (50 mM Tris-HCl, pH 7.0, 150 mM NaCl, 10% D₂O). GD1a is titrated into the LTB solution to prepare the samples with the desired molar ratios of 1:1 and 1:2. The respective HSQC spectra are acquired at 298K. The presence of GD1a does not perturb the signals of LTB TT, indicating no binding between the two molecules.

FIG. 10B shows NMR HSQC spectra of LTB AK titrated with GD1a. The NMR sample containing 15N LTB AK was prepared in an NMR buffer (50 mM Tris-HCl, pH 7.0, 150 mM NaCl, 10% D₂O). GD1a is titrated into the LTB solution to prepare the samples with the desired molar ratios of 1:1 and 1:2. The respective HSQC spectra are acquired at 298K. The presence of GD1a causes the signal of LTB AK to broaden and then disappear, indicating significant binding between the two molecules.

FIG. 11 shows human TLR 2/1 (hTLR 2/1) activity induced by the LTB CPs. HEK 293A cells were genetically transfected to overexpress hTLR 2/1 heterodimer and NF-kB-driven luciferase. The cells were treated with LTB and incubated for 5 hours at 37° C. The binding between LTB and hTLR 2/1 triggers the intracellular signaling pathway to activate NF-κB promoter, leading to the expression of the downstream luciferase gene. The luciferase activity is measured by adding luciferase substrate and the result is used to represent the activity of LTB CPs towards activated TLR 2/1, in which both TT and NN show activity similar to that of WT, while AK, although weak in activity, has the ability to activate hTLR 2/1 heterodimer and downstream NF-κB. Pam3CSK4 is a synthetic lipopeptide that activates the TLR2/1, which is used as positive control.

FIGS. 12A-12E show adjuvant efficacy of LTB TT and NN. (12A) The schedule of intranasal immunization in mice model. Mouse experiments, collecting mouse serum and bronchoalveolar lavage fluid (BALF). ELISA was used to detect neuraminidase (NA)-specific IgG and IgA. BALB/c mice (n=5 per group) were treated with the NA recombinant protein of influenza strain N1 as the immune source, in which N1NA has two mutation sites (N329T and K33IT), adjuvanted with LTB WT, TT or NN, and intranasally immunized three doses. The polyinosinic acid-polycytidylic acid (poly(I:C), a double-stranded RNA analog) was used as positive control for the adjuvant since its ability to trigger antiviral immune responses. ELISAs were conducted to measure (12B) NINA-specific IgG titers in serum and (12C) NINA-specific IgA titers in serum, as well as (12D) NINA-specific IgA titers in BALF and (12E) total IgA titers in BALF.

FIGS. 13A-13C show NA inhibition (NAI) activities of serum and BALFs against influenza strain A/California/04/2009 (H1N1) virus. Enzyme linked lectin assays were used to measured NA inhibition antibody titers as reductions in the NA enzyme activity of Pandemic Influenza A Virus (pH1N1 virus). NAI activity in serum (13A) and in BALF (13B) against A/California/04/2009 (H1N1) virus. (13C) Corresponding half maximal inhibitory concentration (IC₅₀) (referring to the measured half inhibitory concentration of the antagonist), titers were the 50% reduction of virus NA enzyme activity. The results indicate that groups immunized with poly(I:C), LTB WT and its variants TT and NN induced significant NA inhibition titers in serum and BALF compared with unadjuvanted N1NA protein, where the use of TT adjuvant was found to have a higher NAI titer in serum and BALF.

FIG. 14 shows protection against H1N1 virus after adding LTB TT or LTB WT adjuvant and inoculating N1NA recombinant protein. Three weeks after the final immunization, immunized mice (n=6 per group) were infected with 5×LD50 A/PR8 (H1N1) virus intranasally. Survival rates and body weights were monitored daily for 14 days post-infection. It is considered to have no effective protection and cannot survive if body weight loss exceeded 25%.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

As used herein, the term “preventing” or “prevention” refers to stopping or delaying the symptoms of a disease when a drug is administered to a subject who does not have symptoms of disease onset but is at high risk of disease onset.

According to the present invention, E. coli type II heat-labile enterotoxins features the AB₅ structure, consisting of an A-subunit with catalytic activity and a pentameric B-subunit responsible for the binding to host glycoconjugates. The A polypeptide is an enzyme that catalyzes the ADP-ribosylation of the G_{sα} regulatory protein, which upregulates adenylyl cyclase. The B pentamer is involved in the binding of gangliosides, a diverse family of glycolipids found on mammalian cell surfaces. Members of the type II heat-labile enterotoxins have been shown in numerous studies to be effective mucosal adjuvants. It was reported that the non-catalytic B subunits of type II enterotoxins (LTB) interact with Toll-like receptors (TLR). LTB was demonstrated to activate human monocytes or mouse macrophages in a TLR2 signaling pathway. TLR1, which heterodimerizes with TLR2, was also found to be stimulated by LTB. In the absence of an A subunit, LTB initially binds the lipid raft-associated GD1a ganglioside, which recruited TLR2/TLR1 signaling complex to lipid rafts. The GD1a binding facilitates the interaction of LTB with the TLR2/TLR1 signaling complex. This interaction activates the inflammatory transcription factor NF-κB through the intracellular adaptor proteins MYD88 and TRIF downstream of TLR. A consequence of NF-κB activation is the production of inflammatory cytokines, such as TNF-α, IL-1β, IL-6, and IL-8, thereby resulting in the potential for dose-limiting inflammation. This mechanism implies crucial connections with the innate immune system, as GD1a co-receptors facilitate LTB signaling. LTB has been identified as a possible immunomodulatory adjuvant.

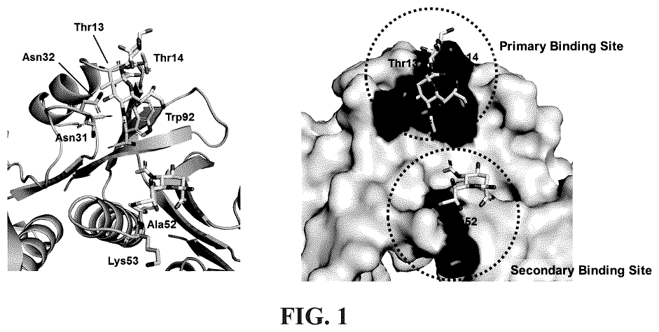

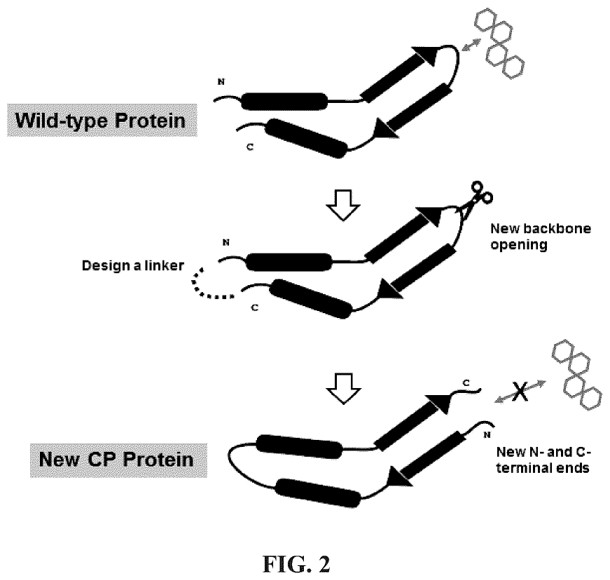

According to the present invention, the Applicant considers the main reason for the side effect caused by LT toxin is the excessive binding to GD1a ganglioside. The adjustment of GD1a binding may alter the induced immunoreactivity. Based on the hypothesis, the Applicant uses the concept of circular permutation (CP) to design the B₅ subunit of type II heat labile enterotoxin (LTB) to effectively modulate the potential adjuvant activity. Previous reported crystal complex structure of LTB and Neu5Ac-nLT that four of the ganglioside ligands bind in a groove between amino acid positions 12-16 of the monomer and amino acid positions 29-36 of the neighboring monomer (FIG. 1). FIG. 2 shows the strategy of using circular permutation (CP) to remove ligand binding. The present invention disconnects the protein peptide chain related to the ligand binding and connects the original terminal ends by a linker to design a GD1a-binding deficient LTB, while retaining the same structure as the original species and maintaining the ability to activate TLR 2/1. The Applicant found that the possible binding sites of LTB and GD1a are both located in the loop, so uses circular permutation to destroy the possible binding sites of GD1a and change the binding affinity of LTB and GD1a.

Thr13 and Thr14 in LTB were the critical residues for GD1a binding. The Thr13 and Thr14 mutations in LTB lead to a decreasing binding ability to GD1a. The deoxy carbon at position 2 of the pyranose ring forms a hydrophobic contact with the methyl group of Thr13, and the carboxylate group of Neu5Ac gains hydrogen bonds from both the side-chain hydroxy group and the main-chain amine of Thr14 (FIG. 1).

In addition, Neu5Ac-nLT forms hydrogen bonds with the side chain of Asn31 and the main chain amine group of Asn32 (FIG. 1).

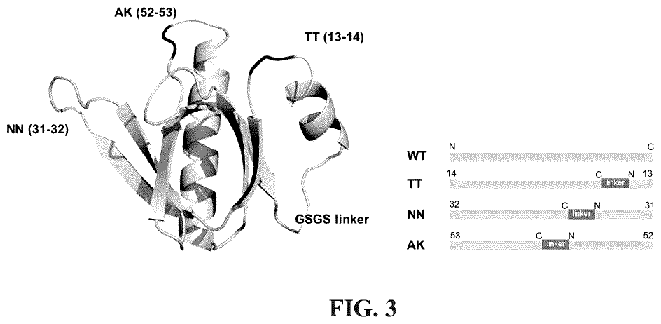

FIG. 3 shows new design of LTB circular permutation constructs. We design three CPs, named TT, NN and AK by disconnecting the corresponding loop and connecting the original terminal ends by a GSGS linker.

FIG. 1 shows the GD1a binding site of LTB. There are two predicted GD1a binding packets on LTB surface (PDB code: 5G3L). The respective critical residues responsible for binding are depicted in FIG. 1. The Applicant believes that LTB and GD1a may have two binding positions. One is the main binding pocket consisting of amino acids 12-14, 30-33, and amino acid 92, and the other is the secondary binding pocket consisting of amino acids 50-55. This is similar to the structure analyzed by NMR in the following examples.

The Applicant considers the primary (major) GD1a-binding site consisting of Arg12, Thr13, Thr14, Ile30, Asn31 and Asn32 (FIG. 1).

Besides, there is another ligand molecule found in the minor site, where it interacts with the residues Arg51, Lys53, Asp54, and Tyr55. The interaction might provide a weaker GD1a binding since the existing interaction is less than that in the previous site. The Applicant considers the positions 51-55 as the secondary (minor) GD1a-binding site.

In the present invention, we design LTB circular permutations (CPs), in which the original N- and C-terminal end is connected and positions of residue 13-14, 31-3 and 52-53 are disconnected to create the new N- and C-termini. A four-amino acid residues (GSGS) (SEQ ID NO:4) linker was added between the original N- and C-termini. Through breaking the backbone linkages at the sites, we enabled to break the GD1a binding site, therefore, reducing the GD1a binding ability. The structural properties of the candidates are evaluated via circular dichroism, NMR, and X-Ray crystallography. The three CPs adopted similar structural fold to the native protein, proving the designs can create native-like LTB variants. However, the three CPs brought distinct structural properties, representing different possibilities to be developed into new protein adjuvant.

Among the three CPs, we identified the LTB variant with decreasing GD1a binding, better structural stability and higher immuno-stimulation comparing to native protein.

The present invention proves the feasibility of using circular permutation to design new LTB adjuvant protein. The newly designed LTB CPs might contribute to the development of mucosal vaccines for influenza and other respiratory disorders. The invention can meet people's urgent needs for novel, effective and safe mucosal adjuvants.

According to the present invention, the amino acid sequence of LTB TT is as shown in SEQ ID NO:1.

According to the present invention, the amino acid sequence of LTB NN is as shown in SEQ ID NO:2.

According to the present invention, the amino acid sequence of LTB AK is as shown in SEQ ID NO:3.

According to the present invention, a 6×His-tag sequence can be conjugated in the C-terminal of LTB to aid purification.

The present invention is further illustrated by the following examples. These examples are provided for illustration only and are not intended to limit the scope of the present invention. The scope of the present invention is shown in the appended claims.

The procedure regarding construct design of LTB circular permutations is as follows. To directly reduce the binding affinity of GD1a, we design LTB circular permutations, which disconnect the bonds between residues 13-14, 31-32 and 52-53. A four-residues (GSGS) linker was added to connect the original N- and C-termini. LTB CPs gene had been synthesized into vector pET22b (+) plasmid. A C-terminal His tag was added for aiding purification, if necessary.

The procedure regarding expression and purification of LTB CPs is as follows. Escherichia coli BL21 (DE3) (Invitrogen) was used for protein expression. The transformation was performed by the heat-shock method and subsequently spread on agar plates. After incubating overnight at 37° C., a single bacterial colony was picked to incubate in small amount of Luria Bertani (LB) medium and shaken overnight at 37° C. Cells were then transferred and cultured in large amount of LB medium with vigorous shaking at 37° C. When OD600 reached 0.6, the cells were induced by 1 mM IPTG for 20 hours at 16° C. The cells were harvested by centrifuging. The cell pellet was resuspended in lysis buffer (50 mM Tris buffer, pH 7.0 and 150 mM NaCl), and was subsequently lysed by using a high-pressure homogenizer. Removing the insoluble fraction from the lysate by centrifuging at 15000 g for 30 minutes, the supernatant was loaded into a column packed with Ni-charged IMAC resin (GE Healthcare). The nonspecific binding proteins were removed by the washing buffer (50 mM Tris buffer, pH 7.0, 150 mM NaCl, and 40 mM imidazole), and the bound His-tagged LTB proteins were eluted with the elution buffer (50 mM Tris buffer, pH 7.0, 150 mM NaCl, and 400 mM imidazole). After concentrating protein with 10,000 Da Amicon membrane ultrafilter (Millipore) at 4° C., Gel filtration was performed to purify the pentameric form of LTB. A HiLoad 16/60 Superdex-75 size exclusion column (GE Healthcare Life Sciences) was equilibrated with the running buffer (50 mM Tris buffer, pH 7.0 and 150 mM NaCl). Under the control of an ÄKTA-FPLC system controller, the flow rate was set up as 1 mL/min.

The procedure regarding mouse immunization and sampling is as follows. Female C57BL/6 and BALB/c mice aged 6 to 8 weeks were procured from the National Laboratory Animal Center, Taiwan. These mice were intranasally immunized with varying doses of recombinant influenza virus N1 neuraminidase (NA) (immune source) along recombinant LTB proteins (10 μg) (adjuvant) or PBS as control where N1NA contains two mutations of N329T and K331T to enhance the cross-reactivity of different strains of virus. Prior to intranasal administrations, mice were under anesthetization and then administered a 30 μl mixture containing NINA (with or without LTB) or a control. All groups of mice were immunized three times at weeks 0, 3, and 6, and sera were sampled at week 8. These mice were euthanized at week 9 for the collection of bronchoalveolar lavage fluid (BALF). All animal procedures were conducted in compliance with the guidelines established by the Laboratory Animal Center of National Tsing Hua University (NTHU) and were reviewed and approved by the NTHU Institutional Animal Care and Use Committee (approval no. 10246).

The procedure regarding analysis of NINA-specific antibody titers is as follows. ELISAs were utilized to quantify antibodies in sera and BALF samples from immunized mice. The 96-well ELISA plates were coated with 100 μl (2 μg/ml of recombinant N1NA protein) and immobilized at 4° C. for 16 to 18 hours. Subsequently, the plates were blocked with blocking buffer (1% BSA in PBS) at 37° C. for 2 hours. Serial dilutions of sera or BALF samples were added to each plate and incubated for 1 hour at room temperature. Following this, HRP conjugated goat anti-mouse IgG antibodies (1:30,000) or HRP conjugated goat anti-mouse IgA antibodies (1:50,000) were added to wells and incubated for an additional 1 hour at 37° C. TMB Chromogen Solution (BioLegend) was mixed for color development, followed by incubation for 15 minutes at 37° C.; the reactions were then halted with 2 N H₂SO₄ and measured by an ELISA reader (OD₄₅₀).

The procedure regarding neuraminidase inhibition assay is as follows. ELISA plates coated with 100 μl (50 μg/ml) of fetuin (Sigma) were incubated at 4° C. overnight. Then, the plates were washed three times with PBST buffer and blocked with PBST buffer for 2 hours. An amount of viruses (H1N1 [A/Puerto Rico/08/1934]) with OD₄₅₀ value of 2 measured by ELISA assay were co-incubated with equal volumes of two-fold serially diluted sera samples for 1 hour at 37° C. and transferred to ELISA plates coated with fetuin for 1 hour at 37° C. After three washes with PBST buffer, 100 μl (2.5 μg/ml) of lectin (Sigma) was added. After 1 hour incubation at room temperature and three washes with PBST buffer, TMB Chromogen Solution was added to the plates and incubated for 15 min in dark. The reactions were terminated by 2N H₂SO₄. The OD₄₅₀ signal was detected by ELISA reader. The serum dilutions that inhibited 50% of NA enzyme activity were defined as IC₅₀ values.

The procedure regarding virus challenges is as follows. Female BALB/c mice aged 6-8 weeks were vaccinated with either three doses of a subunit vaccine or a mock control of PBS. Following the 3rd dose, the immunized mice were intranasally exposed to the H1N1 virus (A/Puerto Rico/08/1934) at a 5-fold mouse median lethal dose (5×LD50). The survival rates and body weights of the mice were monitored daily for 2 weeks, with an individual's weight loss exceeding 25% considered death.

### Example 1

A Strategy of Designing B₅ Subunit of Type II Heat Labile Enterotoxin (LTB) of Escherichia coli CP Variants that the Variants are with Well Structural Stability

Using LTB protein scaffold, the invention is a strategy to design new protein adjuvant by circular permutation. The invention provides three CPs that new backbone disconnections are introduced in the GD1a binding site of Thr13-Thr14 (T13-T14), Asn32-Asn33 (N32-N33) and Ala52-Lys53 (A52-K53). The design will remove/reduce LTB GD1a binding ability.

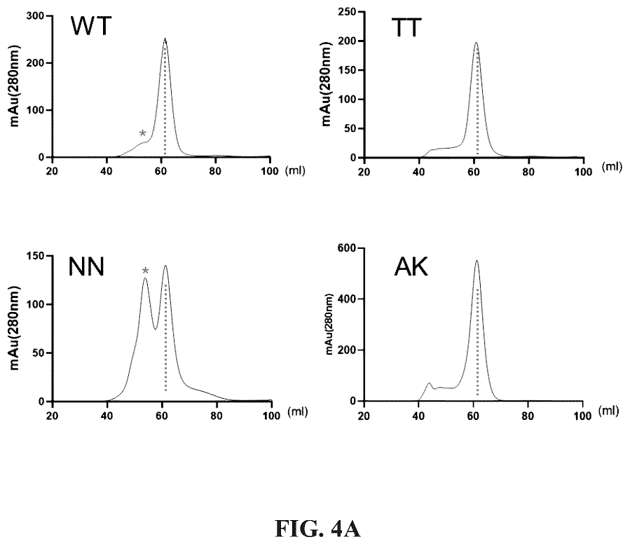

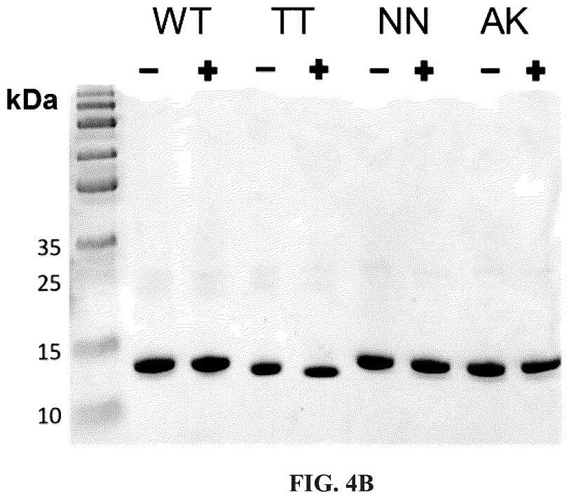

FIGS. 4A and 4B show purification of LTB and the CP constructs. (4A) The FPLC gel filtration profiles of LTB and the CPs. The dotted lines indicate the presence of stable pentamer in LTB WT, TT, NN and AK. Some LTB in WT and NN forms aggregates that elute before the pentamer peaks as indicated by the stars. (4B) SDS-PAGE analysis of purified LTB and the CPs pentamer in the presence and absence of β-mercaptoethanol, showing that excellent purity can be achieved after purification.

FIGS. 4A and 4B show that the three CPs can be well purified and they adopt the same pentamer formation as proved by FPLC gel filtration curves.

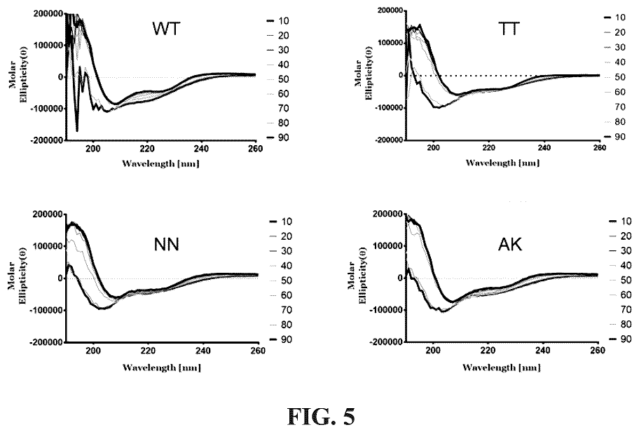

FIG. 5 is circular dichroism (CD) spectra at different temperatures demonstrating LTB CPs sharing the same secondary structural property as WT.

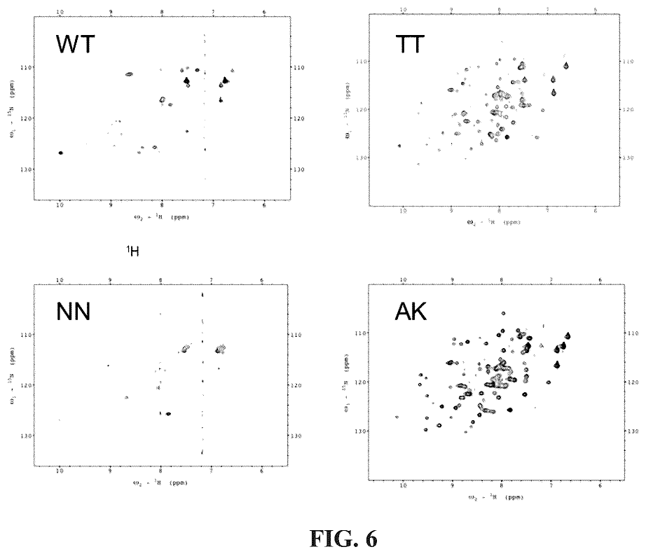

FIG. 6 is NMR HSQC Spectrums displaying the structural stability of LTB CPs. The LTB WT and NN show broadening resonances, indicating that proteins tend to aggregate at high concentrations. The LTB TT and AK show intense and dispersive resonances, indicating correct-folded structure and good structural stability.

FIGS. 5 and 6 indicate that the three CPs adapt the same protein fold as proved by CD and NMR.

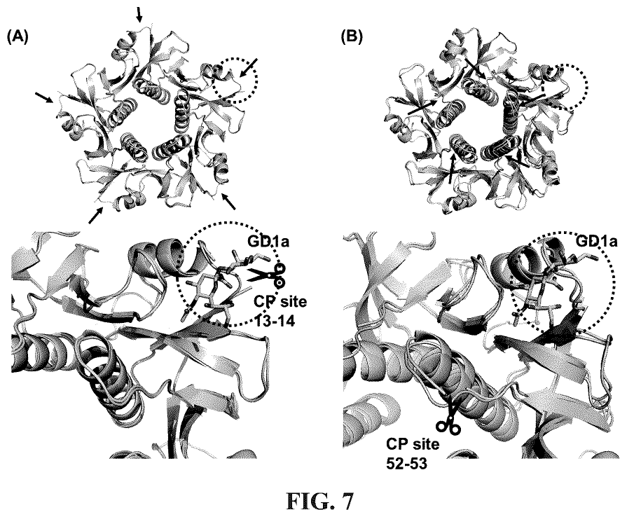

FIG. 7 shows X-ray structures of LTB TT and AK and the comparison with the structure of LTB WT. (7A) The alignment of the structure of LTB TT to the structure of WT shows great structural similarity. The CP sites of LTB TT are indicated by arrows where the representative GD1a main binding site is indicated by a circle. With the GD1a coordinate docking on the LTB TT structure, the GD1a binding packet is disrupted because of protein backbone disconnection. (7B) The alignment of the structure of LTB AK to the structure of WT shows great structural similarity. The CP sites of LTB AK are indicated by arrows where the representative GD1a main binding site is indicated by a circle. With the GD1a coordinate docking on the LTB AK structure, the GD1a binding packet is intact that LTB AK remains the GD1a main binding site, but the secondary binding site is destroyed.

TT has stable structure. Strong NMR HSQC signal reflects its structural stability. We have the crystal structures of TT showing the same structure as WT where the major GD1a binding site is disrupted (FIG. 7A).

AK has stable structure. Strong NMR HSQC signal reflects its structural stability. We have the crystal structures of AK showing the same structure as WT, where the major GD1a binding site is still maintained but the secondary binding site is disrupted (FIG. 7B).

NN contains structural flexibility and the structural behavior is very similar to WT. However, it is more prone to aggregation, protein crystallization cannot be obtained. Comparing to LTB WT, the designed three CPs of the present method all showed either comparable or well structural stability.

### Example 2

The Invention Contains LTB CP Variants that have Modified No GD1a Ganglioside Binding Ability, Such as TT and NN, with No GD1a Binding Ability

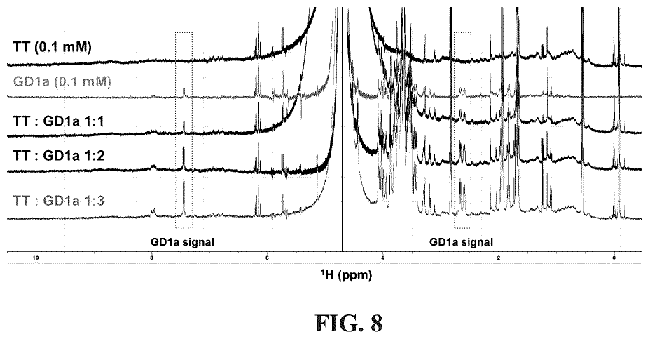

In this embodiment, NMR is used for titration to observe the binding ability to GD1a. FIG. 8 shows NMR 1D ¹H titration experiment. Five NMR 1D ¹H spectra are compared, that are spectra for free LTB TT, free GD1a, TT to GD1a 1:1, TT to GD1a 1:2, TT to GD1a 1:3. The GD1a signals remain the same in the presence of LTB TT. In the subsequent titration, the signal intensity of GD1a is consistent with the dripping multiple, indicating the deficiency of LTB TT for binding GD1a.

We designed LTB variant, TT, which is with no GD1a binding ability (FIG. 8).

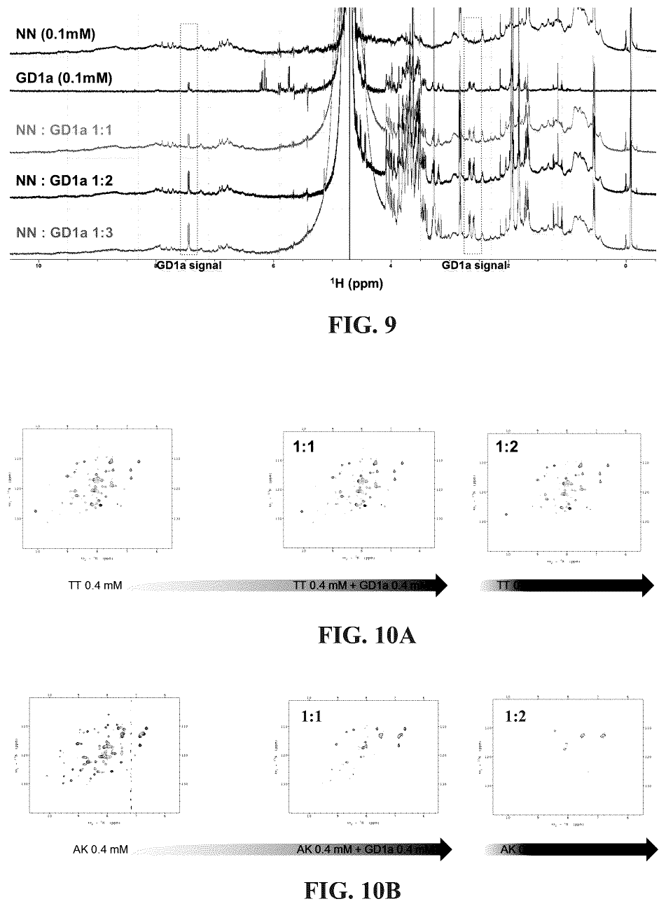

FIG. 9 shows NMR 1D ¹H titration experiment. Five NMR 1D ¹H spectra are compared, that are spectra for free LTB NN, free GD1a, NN to GD1a 1:1, NN to GD1a 1:2, NN to GD1a 1:3. The GD1a signals remain the same in the presence of LTB NN. In the subsequent titration, the signal intensity of GD1a is consistent with the dripping multiple, indicating the deficiency of LTB NN for binding GD1a.

1D ¹H NMR spectra show no binding between GD1a and LTB variant NN (FIG. 9).

FIG. 10A shows NMR HSQC spectra of LTB TT titrated with GD1a. The NMR sample containing ¹⁵N LTB TT was prepared in an NMR buffer (50 mM Tris-HCl, pH 7.0, 150 mM NaCl, 10% D₂O). GD1a is titrated into the LTB solution to prepare the samples with the desired molar ratios of 1:1 and 1:2. The respective HSQC spectra are acquired at 298K. The presence of GD1a does not perturb the signals of LTB TT, indicating no binding between the two molecules.

2D NMR HSQC spectra of TT indicating the presence of GD1a makes no change for TT signals. Again, we prove LTB TT has no binding to GD1a (FIG. 10A).

2D NMR HSQC spectra of NN cannot be performed because of its poor HSQC signals.

FIG. 10B shows NMR HSQC spectra of LTB AK titrated with GD1a. The NMR sample containing 15N LTB AK was prepared in an NMR buffer (50 mM Tris-HCl, pH 7.0, 150 mM NaCl, 10% D₂O). GD1a is titrated into the LTB solution to prepare the samples with the desired molar ratios of 1:1 and 1:2. The respective HSQC spectra are acquired at 298K. The presence of GD1a affects the signal of LTB AK and broadens its spectral line, indicating that GD1a promotes the polymerization of LTB AK, so there is still a binding between the two molecules.

### Example 3

New LTB Variant is the Same as LTB and has Ability to Activate hTLR 2/1 and Trigger Intracellular Signaling Pathway to Activate NF-κB Promoter

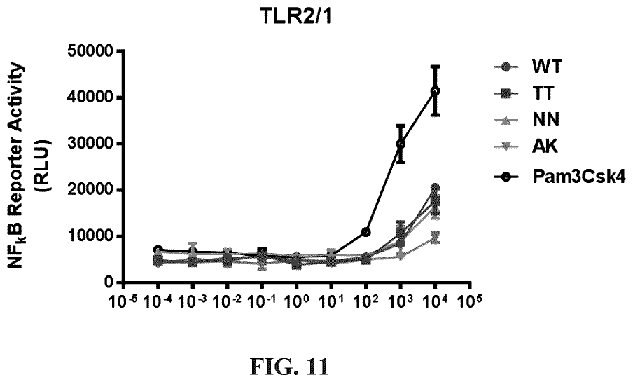

FIG. 11 shows human TLR 2/1 (hTLR 2/1) activity induced by the LTB CPs. HEK 293A cells were genetically transfected to overexpress hTLR 2/1 heterodimer and NF-κB-driven luciferase. The cells were treated with LTB and incubated for 5 hours at 37° C. The binding between LTB and hTLR 2/1 triggers the intracellular signaling pathway to activate NF-κB promoter, leading to the expression of the downstream luciferase gene. The luciferase activity is measured by adding luciferase substrate and the result is used to represent the activity of LTB CPs towards activated TLR 2/1, in which both TT and NN show activity similar to that of WT, while AK, although weak in activity, has the ability to activate hTLR 2/1 heterodimer and downstream NF-κB. Pam3CSK4 is a synthetic lipopeptide that activates the TLR2/1, which is used as positive control.

We have invention of LTB variants, TT, NN and AK. Through activating TLR2/1 pathway, three CPs show comparable activity as NF-κB downstream luciferase gene expression (FIG. 11). Thus, LTB TT, NN and AK are all proved to contain potential, as LTB WT, in being protein adjuvants to enhance immune response.

ELISA is used to determine the binding ability of LTB protein to TLR 2/1, using Pam3CSK4 as a positive control group. It can be seen from the results that TT and NN have almost the same TLR 2/1 binding ability as WT. This shows that TT and NN not only reduce the binding ability to GD1a, but also maintain the binding ability to TLR 2/1.

Compared to other mutations, AK has relatively low TLR1/2 activity, but is not easy to aggregate in aqueous solution, has better structural stability, and retains a certain GD1a binding ability.

### Example 4

New LTB Variant has Better Potential than LTB for Use as Protein Adjuvant

In order to confirm that LTB has antigen adjuvant activity, LTB is used as an adjuvant, and the N1NA protein of the A/California/04/2009 (H₁N1) virus strain (comprising two mutation points N329/K331T) as a nasal spray vaccine to conduct immunization experiments on mice. There are five mice in each group, and different LTB mutations, WT. TT, and NN are used as adjuvants for comparison. Three doses of nasal spray immunization experiments were conducted on mice at 0, 3, and 6 weeks respectively. Blood was collected at the 8th week to obtain the serum, and sacrifice was performed at the 9th week to obtain the bronchoalveolar lavage fluid (BALF). Finally, ELISA was used to detect the IgG and IgA content in the serum and BALF.

PBS and pure antigen were used as the control group, and polyinosinic acid-polycytidylic acid (poly(I:C), a double-stranded RNA analog) was used as the positive control for the adjuvant. Whether in serum or BALF, the anti-N1 IgG and IgA titers of the adjuvanted group were significantly higher than those of the control group. Anti-N1 IgA was not even detected in serum and BALF in the control group. TT produces more anti-N1 IgG and IgA in serum compared with WT and NN, and also produces more anti-N1 IgA in BALF.

As for the total IgA content in BALF, in the adjuvanted group, NN was the highest, WT was the second, and TT was the lowest.

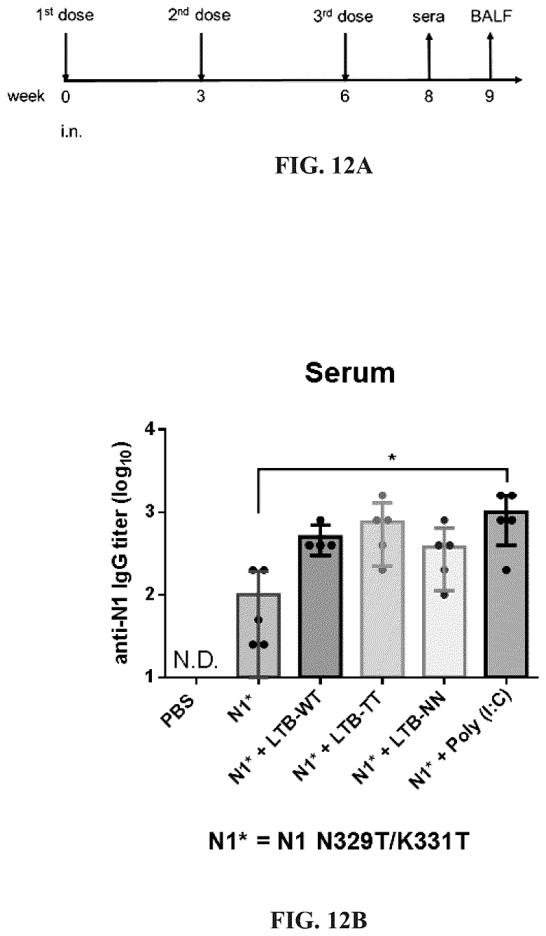

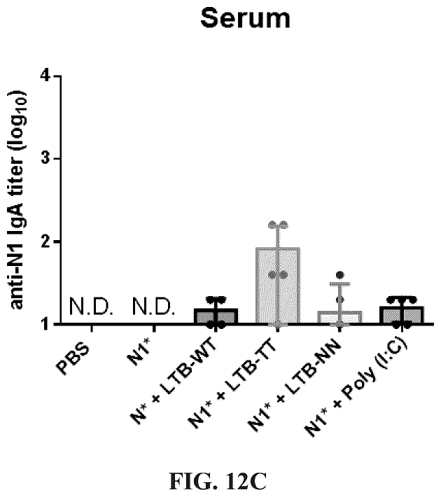

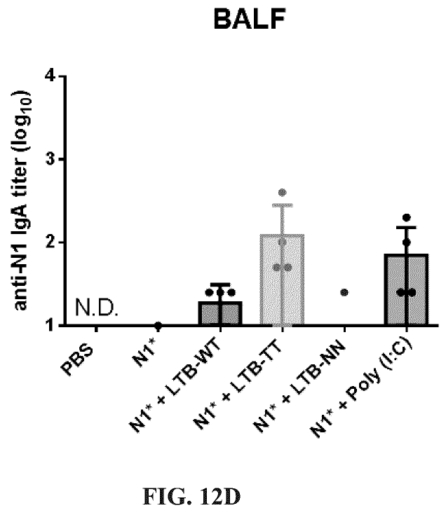

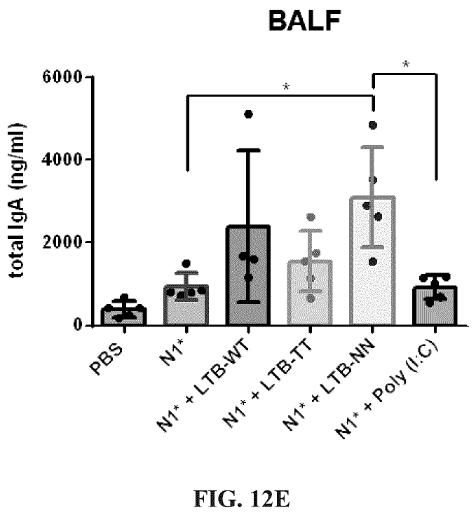

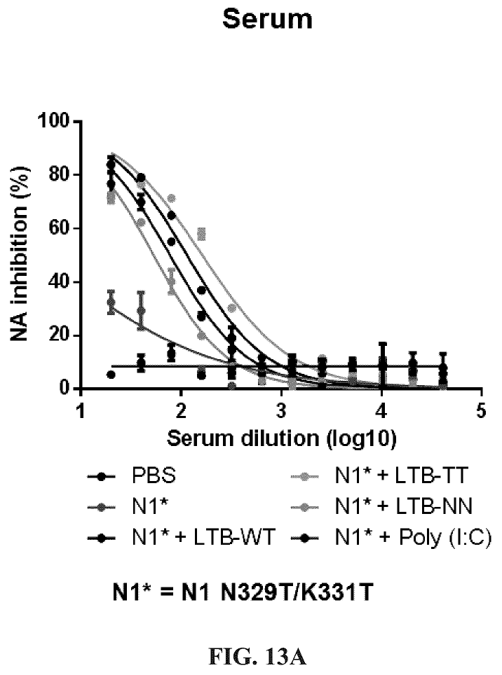

FIGS. 12A-12E show adjuvant efficacy of LTB TT and NN. (12A) The schedule of intranasal immunization in mice model. Mouse experiments, collecting mouse serum and bronchoalveolar lavage fluid (BALF). ELISA was used to detect neuraminidase (NA)-specific IgG and IgA. BALB/c mice (n=5 per group) were treated with the NA recombinant protein of influenza strain N1 as the immune source, in which N1NA has two mutation sites (N329T and K33IT), adjuvanted with LTB WT, TT or NN, and intranasally immunized three doses. The polyinosinic acid-polycytidylic acid (poly(I:C), a double-stranded RNA analog) was used as positive control for the adjuvant since its ability to trigger antiviral immune responses. ELISAs were conducted to measure (12B) NINA-specific IgG titers in serum and (12C) NINA-specific IgA titers in serum, as well as (12D) NINA-specific IgA titers in BALF and (12E) total IgA titers in BALF.

In mouse model, after experienced three doses of intranasal immunization, the immunogen of the influenza virus N1 neuraminidase (NA) in combining with protein adjuvant LTB TT and NN elicits the similar NINA-specific IgG titers in sera comparing to the set of using LTB WT (FIG. 12B).

In mouse model, after experienced three doses of intranasal immunization, the immunogen of the influenza virus N1 neuraminidase (NA) in combining with protein adjuvant LTB TT elicits the significant NINA-specific IgA titers in sera and bronchoalveolar lavage fluid (BALF) (FIGS. 12C and 12D).

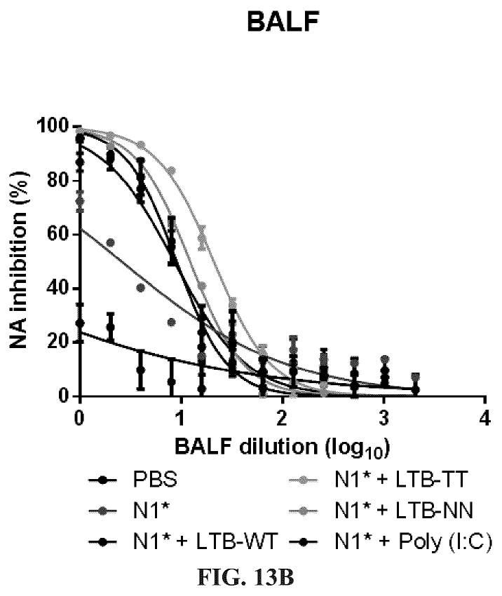

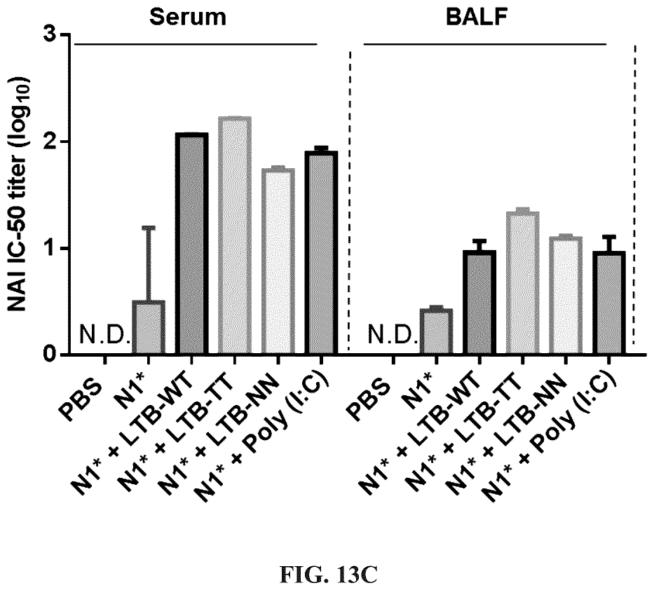

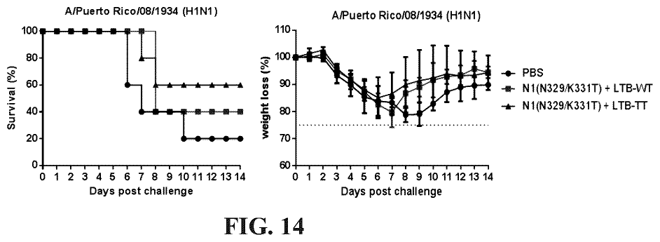

FIGS. 13A-13C show NA inhibition (NAI) activities of serum and BALFs against influenza strain A/California/04/2009 (H1N1) virus. Enzyme linked lectin assays were used to measured NA inhibition antibody titers as reductions in the NA enzyme activity of Pandemic Influenza A Virus (pH1N1 virus). NAI activity in serum (13A) and in BALF (13B) against A/California/04/2009 (H1N1) virus. (13C) Corresponding half maximal inhibitory concentration (IC₅₀) (referring to the measured half inhibitory concentration of the antagonist), titers were the 50% reduction of virus NA enzyme activity. The results indicate that groups immunized with poly(I:C), LTB WT and its variants TT and NN induced significant NA inhibition titers in serum and BALF compared with unadjuvanted N1NA protein, where the use of TT adjuvant was found to have a higher NAI titer in serum and BALF.

To test the neuraminidase inhibitory ability of the antibodies, an enzyme-linked agglutinin assay (ELLA) was used to detect neuraminidase inhibitory antibodies in each immunized group.

Using H1N1 as the virus strain for experiments, it can be found that the serum group with adjuvant added still has a certain antiviral ability even when diluted to 100 times, which is significantly higher than that of the control group. Moreover, TT has a relatively high IC₅₀ titer, while the BALF group has the same trend, and the antiviral ability of the adjuvant added is significantly higher than that of the control group. Moreover, TT also has a relatively high IC₅₀ titer. This experiment proves that among the three adjuvants, TT produces the most specific antibodies and induces the most neuraminidase inhibitory antibodies. The results show that the immune response of TT as an adjuvant to the antigen is higher than that of WT and NN.

The binding between NA antibody and N1 proteins was inhibited by the incorporation of a series of diluted serum and diluted BALF. The results indicate that N1 proteins immunized together with LTB WT and its engineered forms of TT and NN induced significant enhanced inhibition titers in combining with N1 proteins without protein adjuvant (FIGS. 13A-13C).

FIG. 14 shows protection against H1N1 virus after adding LTB TT or LTB WT adjuvant and inoculating N1NA recombinant protein. Three weeks after the final immunization, immunized mice (n=6 per group) were infected with 5×LD50 A/PR8 (H1N1) virus intranasally. Survival rates and body weights were monitored daily for 14 days post-infection. It is considered to have no effective protection and cannot survive if body weight loss exceeded 25%.

N1NA proteins with LTB TT adjuvant was found to have the highest NA antibody inhibition titer. In the protective immunity mouse experiment, LTB TT showed better protective immunity than LTB WT during the challenge of virus (FIG. 14).

In summary, through the results illustrated in the above examples, the present invention uses the LTB protein scaffold to invent a new LTB circular recombinant proteins, and can use this circular recombination to develop new respiratory mucosal protein adjuvants, using circular permutation to design new LTB adjuvants with better structural stability, leveraging circular permutation to maintain the ability to activate TLR2/1 receptors to design novel LTB adjuvants to induce comparable or superior antibody activity. The use of circular permutation to design new LTB adjuvants can reduce the binding ability of GD1a gangliosides, thereby reducing cytotoxicity, designing new LTB adjuvants using circular permutation to induce better immune response stimulation.

The present invention utilizes the concept of circular permutation to design three LTB circular permutation (CP) variants and evaluate their GD1a binding ability and structural stability through nuclear magnetic resonance (NMR) technology. Among new LTB circular permutation variants, TT exhibits excellent structural stability and exhibits superior protein adjuvant activity. NN shows similar structural instability to WT and is easier to aggregate. TT and NN lose their binding ability to GD1a through circular permutation, but still retain their ability to activate TLR 2/1. AK has excellent structural stability and is not easy to aggregate in aqueous solution, retains the binding ability to GD1a, and has low activation ability for TLR 2/1. The circular permutation variants of the present invention give LTB different protein adjuvant activities and can be used for different indications. It is expected to promote the future development of mucosal vaccines, especially for respiratory diseases such as influenza.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A recombinant protein, comprising a B₅ subunit of type II heat labile enterotoxin (LTB) variant of Escherichia coli, wherein the LTB variant comprises at least one amino acid sequence which undergoes circular permutation (CP).

2. The recombinant protein according to claim 1, wherein the circular permutation is performed by disconnecting a specific loop of a B₅ subunit of type II heat labile enterotoxin (LTB) of wild type Escherichia coli and connecting original terminal ends by a GSGS linker.

3. The recombinant protein according to claim 2, wherein positions of residue 13-14, 31-32 or 52-53 of the LTB variant are disconnected to create new N- and C-termini.

4. The recombinant protein according to claim 3, wherein the residue 13-14 is Thr13-Thr14 (T13-T14, TT).

5. The recombinant protein according to claim 3, wherein the residue 31-32 is Asn31-Asn32 (N31-N32, NN).

6. The recombinant protein according to claim 3, wherein the residue 52-53 is Ala52-Lys53 (A52-K53, AK).

7. The recombinant protein according to claim 1, wherein the at least one amino acid sequence which undergoes circular permutation is selected from the group consisting of: SEQ ID NO:1, SEQ ID NO:2, SEQ ID NO:3, and a combination thereof.

8. The recombinant protein according to claim 1, further comprising a 6×His-tag sequence, wherein the 6×His-tag sequence is conjugated in C-terminal of the LTB variants.

9. A method for preparing the recombinant protein according to claim 1, comprising performing circular permutation (CP) on a B₅ subunit of type II heat labile enterotoxin (LTB) of wild type Escherichia coli.

10. The method according to claim 9, wherein the circular permutation is performed by disconnecting a corresponding loop of the B₅ subunit of type II heat labile enterotoxin (LTB) of wild type Escherichia coli and connecting original terminal ends by a GSGS linker.

11. The method according to claim 10, wherein positions of residue 13-14, 31-32 or 52-53 of the LTB variant are disconnected to create new N- and C-termini.

12. The method according to claim 11, wherein the residue 13-14 is Thr13-Thr14 (T13-T14, TT).

13. The method according to claim 11, wherein the residue 31-32 is Asn31-Asn32 (N31-N32, NN).

14. The method according to claim 11, wherein the residue 52-53 is Ala52-Lys53 (A52-K53, AK).

15. A protein adjuvant for a vaccine, comprising the recombinant protein according to claim 1.

16. A method for preventing respiratory disease, comprising administering to a subject in need thereof a pharmaceutical composition comprising an effective amount of the recombinant protein according to claim 1.

17. The method according to claim 16, wherein the respiratory disease is selected from the group consisting of: influenza, severe acute respiratory syndrome (SARS), Coronavirus disease 2019 (COVID-19), and other disease transmitted through respiratory tract.

18. The method according to claim 16, wherein the recombinant protein comprising the at least one amino acid sequence of a combination of SEQ ID NO:1 and SEQ ID NO: 2 does not have or reduces GD1a binding ability.

19. The method according to claim 16, wherein the recombinant protein maintains ability to bind to TLR 2/1.

20. The method according to claim 16, wherein a dosage form of the pharmaceutical composition is intramuscular injection, injection, nasal drops, spray, nasal spray or inhalation.
